(19) United States
(12) Patent Application Publication
(10) Pub. No.: US 2013/0210840 A1
(43) Pub. Date: Aug. 15, 2013

(54) Anti-Cellulite Composition and Method of Treating Cellulite

(71) Applicants: Joseph Grasela, San Diego, CA (US); Joachim Schupp, San Diego, CA (US); Sergio Nacht, Las Vegas, NV (US)

(72) Inventors: Joseph Grasela, San Diego, CA (US); Joachim Schupp, San Diego, CA (US); Sergio Nacht, Las Vegas, NV (US)

(73) Assignee: IMPRIMIS PHARMACEUTICALS, INC., Solana Beach, CA (US)

(21) Appl. No.: 13/703,496

(22) PCT Filed: Jun. 13, 2011
(86) PCT No.: PCT/US11/40132

§ 371 Date: Feb. 21, 2013

Related U.S. Application Data
(60) Provisional application No. 61/354,032, filed on Jun. 11, 2010.

(51) Int. Cl. A61K 31/522; A61K 31/355; A61K 31/07

(52) U.S. Cl. CPC A61K 31/522; A61K 31/07; A61K 31/355
USPC 514/263.34

(57) ABSTRACT

A cellulite-reducing topical composition comprising a lecithin organogel, an ethylene oxide-propylene oxide-ethylene oxide triblock copolymer, caffeine, a retinoid, and optionally at least one vitamin, vitamin derivative or vitamin precursor.

### BACKGROUND

“Cellulite” is a term commonly applied to the lumpy, bumpy, or “orange peel”-like appearance of the skin. Cellulite largely results from changes in the dermis and in subcutaneous tissue contributing to the skin dimpling. Cellulite is observed most commonly in post-adolescent women, especially at the buttocks, abdomen, and thighs and it is unrelated to body weight. At the histological level, cellulite is the result of localized adipose deposits and edema within the subcutaneous tissue. The pathogenisis of cellulite is described, for example, in Leslie A. Baumann M. D., Cosmetic Dermatology, p. 19 (2^{nd} ed. 2009).

Cellulite is generally considered cosmetically undesirable by many women, who attempt to reduce or eliminate the undesirable appearance by topically applying cellulite-reducing (“anti-cellulite”) products. Many products are or have been sold as cellulite-reducing topical compositions and there has been a proliferation of such products. However, the continued demand for improved results indicates the demand and need for more efficacious cellulite-reducing topical compositions. The present invention addresses this need.

### DETAILED DESCRIPTION OF THE INVENTION

As used herein and unless otherwise expressly noted or required by the context, all percentages refer to percentages by weight (wt-%).

As used herein in connection with a measured quantity, for example weight, “about” refers to that variation in the measured quantity as would be expected by one skilled in the art exercising a level of care commensurate with the objective of the measurement and the equipment used, and includes uncertainties that may be introduced by mathematical rounding errors.

The present invention, in one of its embodiments, provides a cellulite-reducing topical composition (anti-cellulite composition) for topical application to the skin of a human. The cellulite-reducing topical composition is efficacious at reducing cellulite and improving the appearance of skin exhibiting cellulite, especially the skin of the buttocks, thighs and abdomen. The efficacy of the cellulite-reducing topical composition of the present invention is demonstrated in end-use testing by users according to the protocol described below.

The cellulite-reducing topical composition of the present invention can be provided in the form of a cream (ointment), a gel, or lotion. Creams are particularly preferred.

The cellulite-reducing topical composition of the present invention, in all of its embodiments, includes, as an essential component, caffeine (3,7-dihydro-1,3,7-trimethyl-1H-puridine-2,6-dione). The caffeine is present in the cellulite-reducing topical composition at about 1 wt-% to about 5 wt-%, preferably about 3 wt-%.

The cellulite-reducing topical composition of the present invention, in all of its embodiments, also includes, as an essential component, a lecithin organogel that is obtained by combining a lecithin with an alkyl ester of a fatty alcohol and, optionally, other ingredients that do not interfere with or disrupt formation of the lecithin organogel.

The lecithins useful in obtaining the lecithin organogel useful in the practice of the present invention are phospholipids that are fatty acid diesters of the choline ester of glycerophosphoric acid. Such fatty acid diesters of the choline ester of glycerophosphoric acid are commonly referred to as phosphatidyl cholines.

Soy lecithin is a preferred fatty acid diester of the choline ester of glycerophosphoric acid for use in the practice of the present invention, but other phosphatidyl cholines can be used.

The alkyl esters of fatty alcohols (fatty acid esters) useful in obtaining the lecithin organogels useful in the practice of the present invention are alkyl esters of linear or branched, preferably linear, saturated or unsaturated, preferably saturated, aliphatic fatty acids having 12-22, preferably 14-18, carbon atoms.

The alkyl group of the alkyl esters of fatty alcohols is an alkyl group having 1 to about 5 carbon atoms and, when it contains 3 or more carbon atoms, can be and preferably is a branched alkyl group. Isopropyl palmitate (prop-2-yl hexadecanoate) is a particularly preferred alkyl ester of a fatty alcohol for use in obtaining the lecithin organogel.

The fatty acid diester of the choline ester of glycerophosphoric acid and the alkyl ester of a fatty alcohol are used in a weight ratio of about 2.5:1 to about 0.3:1 to obtain the lecithin organogel. A weight ratio of about 1:1 is preferred.

The lecithin organogel useful in the practice of the present invention can, and in preferred embodiments does, include an essential oil. Essential oils, or aetherolea, are hydrophobic liquids that are extracts or steam distillates of an expression of plant matter, especially plant matter that is a fruit. Citrus medica limonium is a preferred essential oil that can be included in the lecithin organogel useful in the practice of the present invention. When used in making the lecithin organogel, the essential oil is used in an amount such that the concentration in the cellulite-reducing topical composition is about 0.03 to 0.10 wt-%.

The cellulite-reducing topical composition of the present invention, in all of its embodiments, further includes, as an essential component, an ethylene oxide-propylene oxide-ethtyene oxide triblock copolymer (EO/PO/EO triblock copolymer). EO/PO/EO triblock copolymers have a central block of propylene oxide (PO) units flanked on both ends with blocks of ethylene oxide (EO) units. The EO/PO/EO triblock copolymers useful in the practice of the present invention are commonly referred to as “poloxamers”.

Preferred EO/PO/EO triblock copolymers have an EO content of 55 wt-% to 85 wt %, more preferably 65 wt-% to 75 wt-%, still more preferably of about 70 wt-%.

The central PO block of the EO/PO/EO triblock copolymers useful in the practice of the present invention is made-up of about 55-85, preferably 65-85, still more preferably about 70 PO repeating units or residues. Pluronic® poloxamers available from BASF Corp., Florham Park, N.J., are examples of EO/PO/EO triblock copolymers useful in the practice of the present invention. Pluronic® 407 is a preferred EO/PO/EO triblock copolymer.

The cellulite-reducing topical composition of the present invention can, and in preferred embodiments does, include a retinoid.

Retinoids are well known to those skilled in the art of formulating topical dermatological compositions. Retinoids exhibit the pharmacological activity of all trans retinol and share, as a common structural feature, a β-ionone-type ring (2,6,6-trimethylcyclohen-1-ene) having a multiply unsaturated alkyl side chain at the 1 position of the ring. Although retinol is a preferred retinoid for use in those embodiments of the present invention in which a retinoid is included, other retinoid derivatives can also be used, including retinyl palmitate or retinyl linoleate.

When used, the retinoid is present in the cellulite-reducing topical composition at about 0.1 wt-% to about 3.0 wt-%, preferably about 0.2 wt-%.

The cellulite-reducing topical composition of the present invention can, and in preferred embodiments does, still further include one or more vitamins or vitamin derivatives or precursors. A vitamin derivative is a vitamin that has been modified by, for example, salification or esterification, to improve resistance to chemical degradation, for example oxidation, to alter its solubility properties, or both. Vitamin derivatives useful in the practice of those embodiments of the present invention that include the vitamin E and C derivatives tocopherol acetate and ascorbyl palmitate. An example of a vitamin precursor useful in this invention is panthenol, a pro-Vitamin B₆.

When used, vitamins or vitamin derivatives are present in the cellulite-reducing topical composition of the present invention at about 1.0 wt-% to about 6 wt-%, preferably at about 2.5 wt-%.

The cellulite-reducing topical composition of the present invention, in all of its embodiments, includes a dermatologically acceptable carrier base. In the context of the present application, a carrier base is dermatologically acceptable if it does not interfere with the cellulite-reducing properties of the cellulite-reducing topical composition and does not cause an adverse reaction (e.g. contact dermatitis) in a majority of the users of the composition.

The dermatologically acceptable carrier base is selected according to the desired final form of the topical composition (cream or ointment, gel, lotion, and the like) from the types of carrier bases known in the art for topical application of active ingredients.

In preferred embodiments, the cellulite-reducing topical composition of the present invention is in the form of a cream and the dermatologically acceptable carrier base is a water-in-oil emulsion emollient carrier base. Dermabase™ cream, available from Paddock Laboratories, Inc., Minneapolis, Minn., is an example of a preferred dermatologically acceptable cream carrier base.

The cellulite-reducing topical composition of the present invention can, and in preferred embodiments does, include further components, in particular other skincare active ingredients, preservatives, stabilizers, and chelating or sequestering agents.

Non-limiting examples of stabilizers and preservatives known to persons of skill in the art include BHT, BHA, potassium sorbate, citric acid, and sorbic acid. Disodium EDTA is an example of an optional chelating or sequestering agent.

Additional skincare active ingredients that may be added to the compositions of the present invention have one or more of the following properties: anti-oxidant; anti-edematous; anti-inflammatory; stimulate lipolysis; reduce phosphodiesterase activity and/or levels; improve/increase microvascular perfusion; promote production of collagen and/or elastin (or prevent their degradation); thickening the dermis; prevent fat herniation into superficial tissue; prevent or destroy free-radical formation; inhibit the release of acetylcholine; help reduce the appearance of skin discoloration; anti-estrogen activity; anti-androgen activity; anti-collagenase activity; and/or increase the synthesis of sirtuins. Non-limiting examples of such active ingredients include: antioxidants, including but not limited to resveratrol, extracts of one or more of agai, blueberry, papaya, pineapple, chofitol; rutin; proanthocyanidins; extracts of Centella asiatica, especially triterpenes (asiatic acid, asiaticoside and madecassic acid); ursolic acid; Ginkgo biloba dimeric flavonoids; dipalmitoyl hydroxyproline; growth factors; short-chain peptides having from 2 to 12 amino acids, preferably acylated, that increase the expression of genes that code for one or more of collagen or elastin; short-chain peptides having from 2 to 12 amino acids, preferably acylated, that inhibit the expression of one or matrix metalloproteinases, preferably MMP-1, MMP-2, MMP-8, MMP-9 and/or MMP-13; phytotoxins and/or phycotoxins, including red algae extract; Coleus forskolii extract; beta-adenergic agonists; alpha-adrenergic antagonists; methylxanthines and its derivatives, including but not limited to, aminophylline and its derivatives, theophylline and its derivatives, including theophylline acetate; pentoxyphilline; and carnitine and its derivatives, including acetyl carnitine.

The cellulite-reducing topical composition of the present invention is prepared in a four-step process: preparation of a lecithin organogel first premix, preparation of a EO/PO/EO triblock copolymer second premix, preparation of a third premix, and preparation of the cellulite-reducing topical composition. The first two steps can be performed in any order.

In a first step, a fatty acid diester of the choline ester of glycerophosphoric acid and the alkyl ester(s) of fatty alcohols are combined, in any order, optionally with agitation to promote mixing, and held in the mixed state, optionally with agitation, for a period of about 8 to 32 hours to obtain the lecithin organogel that is a first premix. The skilled artisan will know to attenuate the intensity of mixing to not interfere with formation of the organogel.

In a second step, the EO/PO/EO triblock copolymer and, optionally, a preservative such as potassium sorbate, is combined with water, optionally with agitation, to promote mixing, to obtain a second premix. The ratio of EO/PO/EO triblock copolymer to water is about 0.07:1 to about 0.25:1.

In a third step, the first premix, the second premix, and, if used, the retinoid, vitamin or vitamin derivative, and further ingredients, are combined, in any order, preferably with agitation, to obtain a homogeneous third premix. The skilled artisan will know to attenuate the intensity of agitation, when used, to provide sufficient mixing without disrupting or destroying the lecithin organogel of the first premix, to obtain a third premix.

In a fourth step, the third premix is compounded with the dermatologically acceptable carrier base and, optionally, other ingredients. The skilled artisan will know to select the compounding equipment for this fourth step according to the type of dermatologically acceptable carrier base used and the form of the cellulite-reducing topical composition to be provided.

For example, when a cream or ointment product is to be provided, a cream carrier base is used, and compounding equipment of the well-known homomixer type can be used. Important is that the compounding equipment chosen not provide an intensity of mixing such that the lecithin organogel is destroyed or disrupted, interfering with the cellulite-reducing function of the cellulite-reducing topical composition of the present invention. Otherwise, the choice of compounding equipment is left to the judgment, and is within the ken, of the skilled artisan.

The composition of the cellulite-reducing topical composition of the present invention can be as follows:

| Ingredient | INCI Name | wt-% | (Range) |
|---|---|---|---|
| Dermabase ™ | Water (and) | 72.50 | (61.5-77.50) |
| Cream | Petrolatum (and) | | |
| | Mineral Oil (and) | | |
| | Cetostearyl Alcohol | | |
| Water | Water (Aqua) | Q.S. | |
| Ascorbyl Palmitate | Ascorbyl Palmitate | 2.00 | (1.00-5.00) |
| Lecithin Soya | Lecithin | 4.50 | (2.25-6.25) |
| Powder | | | |
| Isopropyl Palmitate | Isopropyl Palmitate | 4.50 | (2.50-6.50) |
| Caffeine Anhydrous | Caffeine | 3.00 | (1.00-5.00) |
| Powder | | | |
| Pluronic F-127 | Poloxamer 407 | 1.80 | (1.00-2.75) |
| | (BASF) | | |
| Vitamin E Acetate | Vitamin E Acetate | 0.50 | (0.10-1.00) |
| Retinol 50 C (BASF) | Retinol (and) | 0.40 | (0.100-2.00) |
| | Polysorbate 20 | | |
| BHT FCC | Butylated | 0.25 | (0.15-0.50) |
| | Hydroxytoluene | | |
| Citric Acid USP-FCC | Citric Acid | 0.20 | (0.10-0.40) |
| Lemon Oil Liquid | Citrus Medica | 0.06 | (0.03-0.10) |
| | Limonum (Lemon) | | |
| Sorbic Acid NF FCC | Sorbic Acid | 0.020 | (0.005-0.05) |
| Potassium Sorbate | Potassium Sorbate | 0.015 | (0.005-0.040) |
| Dissolvine NA2S | Disodium EDTA | 0.010 | (0.001-0.025) |

The cellulite-reducing topical composition of the present invention can be packaged in packaging suitable for its physical form.

In another embodiment, the present invention provides a method for treating cellulite, or for ameliorating the physically observable manifestations of cellulite (i.e. “orange peel”). According to this embodiment, compositions of the invention will be applied topically on body areas affected by localized adiposities and/or cellulite, for time periods ranging from several days to several months, depending on the severity of the condition to be treated, with frequency of 1-2 applications per day.

The method of the present invention includes the step of applying to human skin, especially the skin of the buttocks, thighs and abdomen of a human, especially a female human, an amount of the cellulite-reducing topical composition of the present invention sufficient to apply about 1 to about 10 milligrams per square centimeter of skin of caffeine.

The present invention, in one of its embodiments, is illustrated by the following non-limiting example.

### Formulation Example

### Preparation of Lecithin Organogel First Premix

| Ingredient | Amount | |
|---|---|---|
| Lecithin Granules | 10.00 | g |
| Sorbic Acid NF | 0.05 | g |
| Citrus medica limonium | 0.15 | ml |
| Isopropyl palmitate | 0.80 | ml |

The above ingredients are combined and allowed to rest for 14 hours to obtain a lecithin organogel first premix.

### Formulation Example

### Preparation of EO/PO/EO Triblock Copolymer Second Premix

| Ingredient | Amount |
|---|---|
| Poloxamer 407 EO/PO/EO triblock copolymer | 200.00 g |
| Water | 800.00 g |
| Potassium sorbate | 2.00 g |

### Formulation Example

### Preparation of Third Premix

| Ingredient | Amount (grams) |
|---|---|
| Retinol 50 C | 0.40 |
| Tocopherol acetate | 0.50 |
| First premix | 9.00 |
| Second premix | 9.10 |

Retinol, vitamin E acetate, first premix, and second premix are combined and mixed thoroughly to obtain a third premix.

### Formulation Example

### Preparation of Cellulite-Reducing Topical Composition

| Ingredient | Amount (grams) |
|---|---|
| Ascorbyl palmitate | 5.00 |
| Caffeine (anhyd.) | 3.00 |
| Citric acid | 0.02 |
| BHT | 0.25 |
| Disodium EDTA | 0.01 |
| Third premix | 18.00 |

Water-in-oil emollient cream carrier base 72.50
(Dermabase™ cream)

The ascorbyl palmitate, caffeine, citric acid, BHT, and disodium EDTA are combined and triturated, and the triturate is thoroughly mixed with the third premix. The resulting mixture is combined with the carrier base and thoroughly blended to obtain a cellulite-reducing topical composition that is a cream.

### Clinical Efficacy Example

The efficacy of the cellulite-reducing topical composition of present invention is illustrated by administration of the above-prepared cellulite-reducing cream topical formulation on the thighs of middle-aged women according to the following protocol:

A group of ten female subjects, 50 to 65 years of age, are recruited to participate in an eight-week clinical study. Inclusion criteria require that the subjects be in good health, with no skin disease, except for adult acne or mild rosacea. Women who are currently being treated with systemic retinoids, or have been under treatment with systemic retinoids within six months of the Study start date were not eligible to participate. Likewise, women who 30 days prior to the scheduled start date had been treated with systemic antibiotics, antihistamines, or systemic steroids were excluded. In addition, women who are currently or have been under treatment within three months with topical retinoids or within two weeks with topical steroids, keratolytics, antimicrobials, or acne products were not eligible to participate. Additionally, women having known allergy to or skin irritations from retinol, vitamin C, vitamin E, caffeine, sodium lauryl sulfate, soy lecithin, parabens or urea were excluded.

The participants are provided with the above cellulite-reducing cream topical formulation (the “test product”), which is applied twice daily, morning and evening, per instructions in accordance with the approved protocol for the duration of the Study. Neutrogena soap bars are supplied by the Investigator and are used by the participants for washing throughout the study. With the exception of water-washable facial makeup, no other soap, cleanser, or topical product is used during participation in the Study.

Clinical evaluations are performed at baseline, then at 14 days, 28 days and 56 days after initiating the product application regimen. In addition, medical grade color photographs of the subjects are taken at each Study visit. More particularly, photographs are taken of the outer thigh, from hip to knee, and a close-up shot of the skin on the thigh, to show fine lines and wrinkles superimposed on the dimpling and undulations characteristic of cellulite. A manual “pinch test” is performed on each subject to assess skin looseness. Skin firmness and resilience by indentometry are measured. Subjects also provide a self-assessment of cellulite severity and improvement.

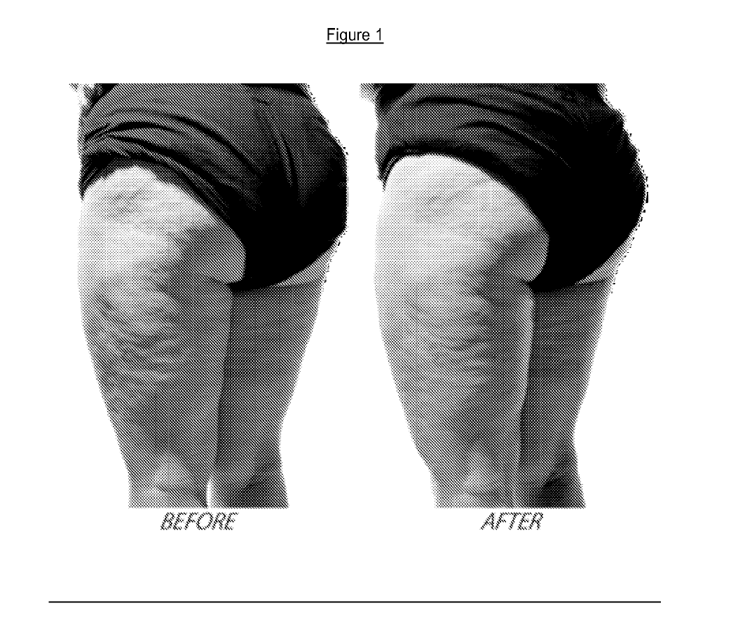

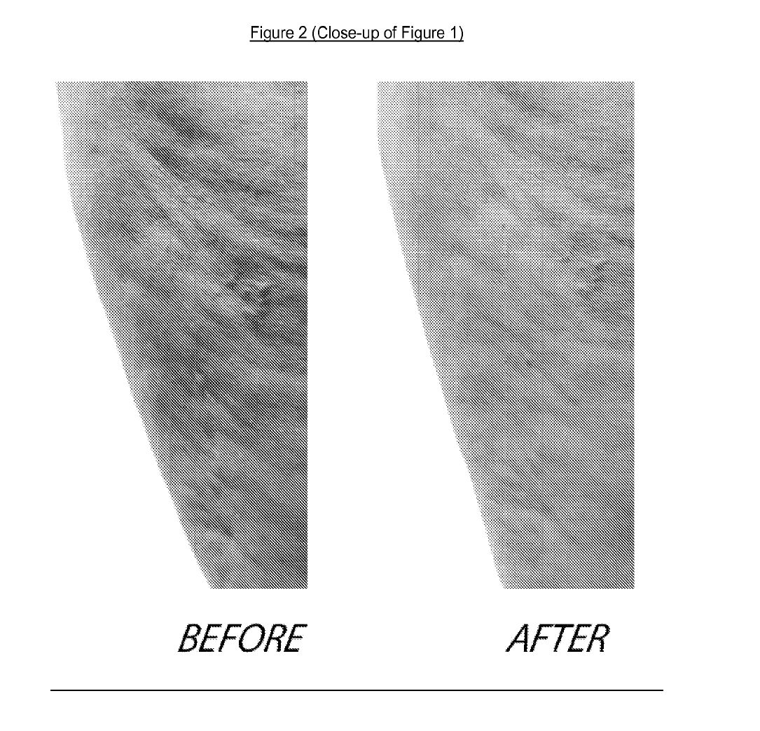

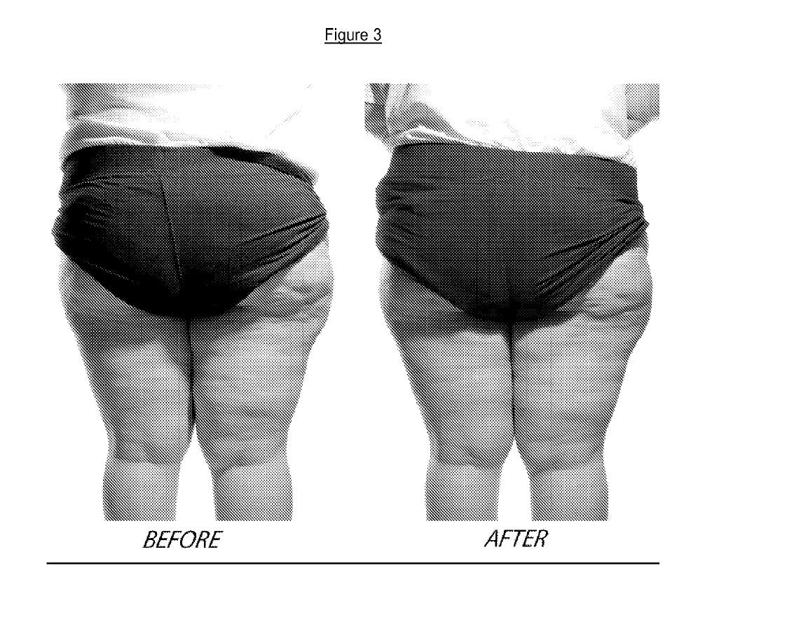

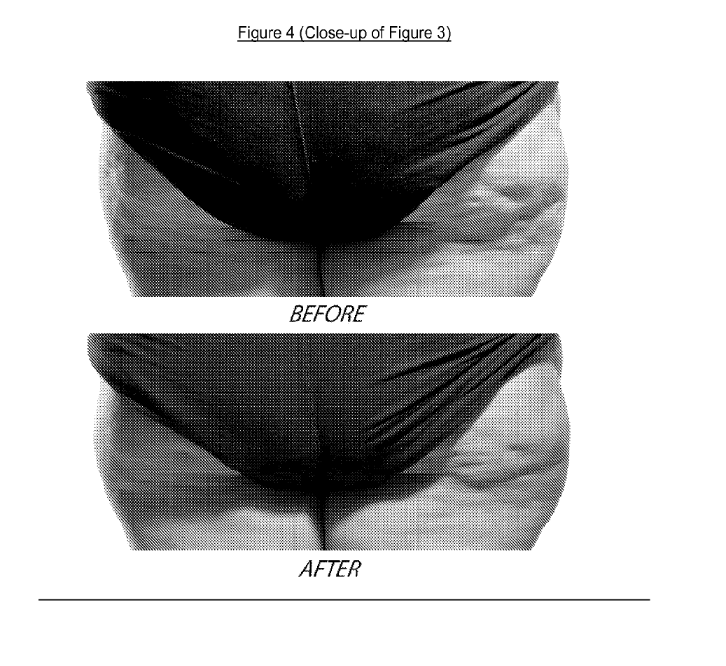

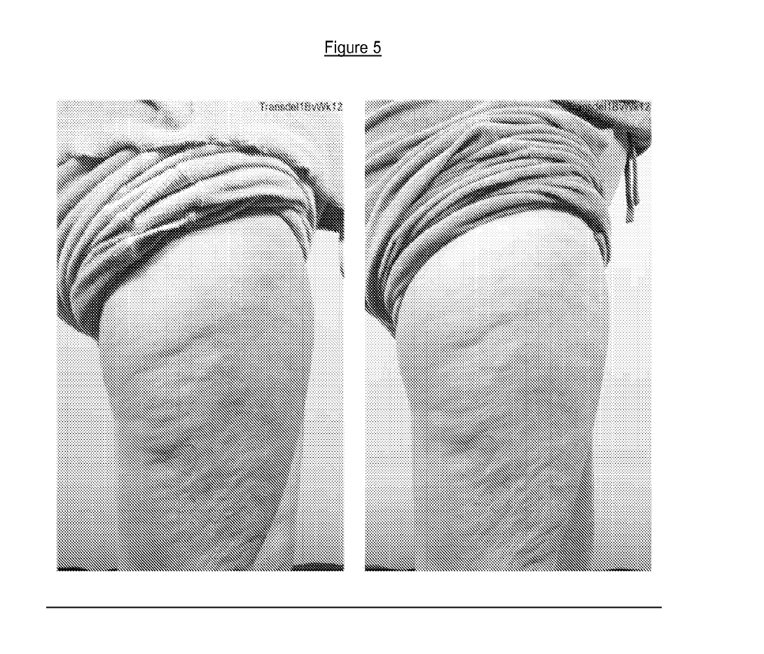

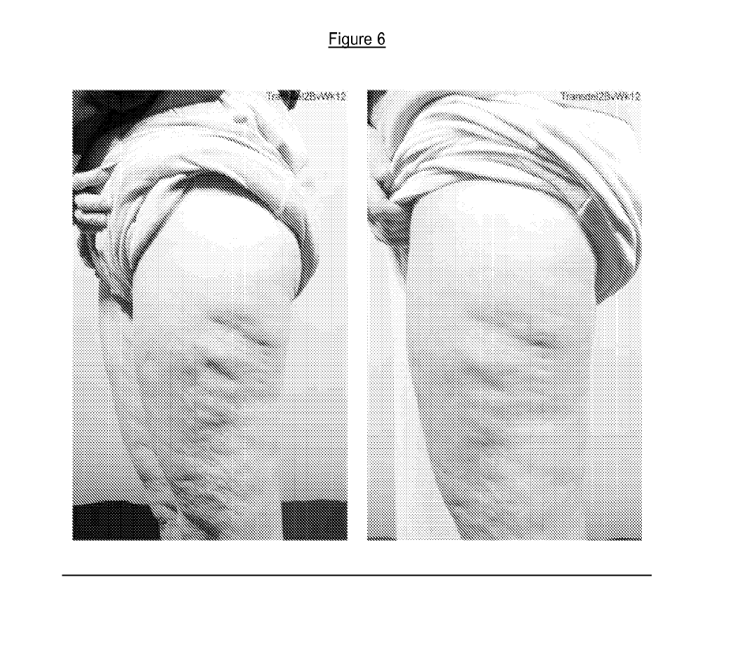

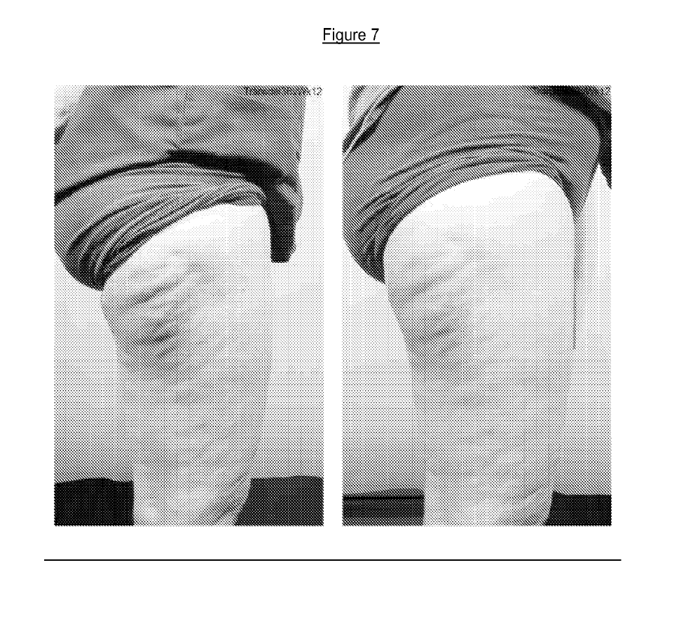

Throughout and at the culmination of the Study, participants are observed and measured to have one or more of the following improvements: increased skin visco-elastic behavior (elasticity and firmness) as measured, for example, with a twistometer; decreased number and depth of fine lines and wrinkles as measured, for example, by Silflo replica models; reduced appearance of cellulite ripples (hills and valleys on the lateral thighs); reduced circumference of thighs; reduced appearance of lumpy, bumpy, or “orange peel”-like skin. Improvements of the type shown in FIGS. 1-7 are observed.

What is claimed:

1. A cellulite-reducing topical composition comprising:
(a) about 5% to about 13% by weight of a lecithin organogel,
(b) 1% to 3% by weight of a ethylene oxide-propylene oxide-ethylene oxide triblock copolymer,
(c) 1% to 5% caffeine, and
(d) a dermatologically acceptable carrier base.

2. The cellulite reducing topical composition of claim 1 further comprising about 0.1% to about 3.0%, by weight, of a retinoid.

3. The cellulite reducing topical composition of claim 2 wherein the retinoid is retinol.

4. The cellulite reducing topical composition of claim 1 further comprising about 0.1% to about 6.0%, by weight, of at least one vitamin, vitamin derivative or vitamin precursor.

5. The cellulite reducing topical composition of claim 4 wherein the at least one vitamin derivative is tocopherol acetate, ascorbyl palmitate, or a mixture thereof.

6. The cellulite reducing topical composition of claim 1 wherein the organogel is comprised of soy lecithin and isopropyl palmitate.

7. The cellulite reducing topical composition of claim 6 wherein the organogel further comprises at least one anti-cellulite active ingredient selected from the group of: anti-inflammatory agents; extracts of Centella asiatica, Coleus forskolii, Ginkgo biloba; ursolic acid; short-chain peptides having from 2 to 12 amino acids; MMP-inhibitors; theophylline and derivatives thereof; carnitine and derivatives thereof.

8. The cellulite reducing topical composition of claim 1 wherein the ethylene oxide-propylene oxide-ethylene oxide triblock copolymer comprises about 70%, by weight, ethylene oxide.

9. A method of reducing cellulite in human skin, and the visual manifestations thereof, comprising the step of applying to the skin an amount of the cellulite reducing topical composition of any one of claims 1 to 8, the composition being applied at from about 30 to about 300 mg/cm², sufficient to deliver from about 1 to about 10 milligrams of caffeine per square centimeter of skin surface.
